(19) United States
(12) Patent Application Publication
(10) Pub. No.: US 2007/0231396 A1
(43) Pub. Date: Oct. 4, 2007

(54) Medication spray formulation

(76) Inventor: Charles D. Ray, Santa Barbara, CA (US)

Correspondence Address: DICKE, BILLIG & CZAJA, FIFTH STREET TOWERS, 100 SOUTH FIFTH STREET, SUITE 2250, MINNEAPOLIS, MN, 55402, US

(21) Appl. No.: 11/392,403

(22) Filed: Mar. 29, 2006

(51) Int. Cl. A61K 36/899; A61K 36/47; A61K 31/56; A61K 31/545; A61K 9/50

(52) U.S. Cl. 424/490; 424/731; 424/750; 514/200; 514/179

(57) ABSTRACT

Formulations for delivery of a pharmaceutical agent include a thixotropic agent. The pharmaceutical agent may be slowly released from a hydrogel or microcapsule in the spray formulation. Various formulations of reasonable thixotropic and hydrogel media are useful. Droplet size and thixotropy determine the distribution of the material along body surfaces and tissues such as the respiratory passages, and limit the displacement, running or dripping of the sprayed medium from its intended sites.

### FIELD OF THE INVENTION

The invention relates to thixotropic formulations for spray media delivery to body surfaces and tissues such as nasopharyngeal passages. The thixotropic effect allows for good fluidity during spraying but prevents running or drippage loss of the medication from the body surface so sprayed. Additionally, a suitable hydrogel or microencapsulation is provided into which a medication is carried and slowly released into absorptive tissues receiving the spray.

2. Description of the Relevant Art

Atomization or jet spray of fluid media to deliver various compounds, such as emollients, thin creams, oils, aqueous or powdered substances or medication to tissues, particularly within the nasopharyngeal mucosa or other body surfaces, have been known and practiced for centuries. Various means to deliver a burst or steady flow of media by a repelling agent, usually air, as by blowing through a hollow reed or using a compressible bulb or by a pressurized compound or gas, are well described in existing art. The expelled material may be atomized, fluidized or ejected in a stream. In order to obtain an optimized dispensing of the medium to be sprayed, several interdependent functions must be considered. These are related to the expulsion force, the nozzle or spray jet and the medium to be sprayed. For the spraying to continue with satisfaction, the following must be optimized: applied ejection pressure, velocity, fluidity (viscosity), turbulence and surface tension of the suspending medium, as well as characteristics of the dissolved or suspended particulates in the expellant medium. Also important is the structure of the spray nozzle, or ventura. In the case of plain or medicated substances sprayed along the nasopharyngeal passages, additional parameters of consequence are the substance particle or droplet size, the atomizer nozzle and suspension medium. They must all be optimized relative to the delivery means, usually a hand operated rubber bulb. Optimizing the media, medication suspension and dispensing device must be performed by a trial and error method.

Further, for many applications the fluidity (viscosity) of the medium must be relatively low to permit easy spray atomizing or dispersal as micro particles. After the medium is in place, a preferred medium should not run down from or drip out or off of the sprayed surface(s). These two characteristics (spray fluidity and non-drip) are found in non-Newtonian thixotropic fluids. A Newtonian fluid shows a viscosity that is directly related to the velocity of motion of the fluid. Water, air and most aqueous solutions typify such fluids. Thixotropic fluids show a viscosity that falls with increased velocity, becoming thicker when the fluid medium is quiet or still. Unfortunately, much of the usual sprayed, watery material will coalesce and drain by gravity, especially when further diluted into the mucous, serous fluid or sweat present on body surfaces. The thixotropic effect reduces such coalescence and drainage.

It would be very desirable to provide a means to dispense a pharmaceutical agent under low pressure using a medium that has a low sheared viscosity, and which, after spraying and deposition on the body surface, returns to the unsheared viscosity and does not drip or run. Such a product would be welcomed by both manufacturers of health care products and consumers of such products.

### SUMMARY OF THE INVENTION

The present invention provides a thixotropic fluid media composition that is capable of being sprayed onto a patient's tissue (e.g., skin, wound, nasal cavity/mucous membrane), and generally consists of a primary carrier component (or “suspended medium”) and a thixotropic agent.

Specifically, the invention provides an aqueous fluid suspension for delivery of a pharmaceutical agent, said suspension having a sheared viscosity and an unsheared viscosity, wherein said sheared viscosity is less than about 200 centipoise, the suspension being sprayable when sheared, and wherein said unsheared viscosity is at least about 5 times greater than said sheared viscosity, and the suspension returns to its unsheared viscosity within about 20 seconds after shearing, the suspension comprising said pharmaceutical agent and a carrier therefore, said carrier comprising a thixotropic agent.

In one embodiment, the invention provides an aqueous fluid suspension for delivery of a pharmaceutical agent, said suspension having a sheared viscosity and an unsheared viscosity, wherein said sheared viscosity is less than about 200 centipoise, the suspension being sprayable when sheared, and wherein said unsheared viscosity is greater than about 400 centipoise said suspension comprising said pharmaceutical agent and a carrier therefore which includes a thixotropic agent,

In another embodiment, the thixotropic agent comprises a hydrogel.

It will be recognized that the suspension in accordance with the present invention may have a variety of different carrier formulations, each specifically selected to generate a desired end effect in the patient. Thus, the present invention is in no way limited to a particular carrier formulation (or desired end use), although in some embodiments described below, a hydrogel material is included with the carrier component. Rather, the present invention is premised upon the provision of the thixotropic agent in combination with the primary carrier component to achieve the overall reduced viscosity upon spraying to the targeted tissue, thereby minimizing undesired “dripping” of the sprayed compound from the targeted tissue.

As used herein, these terms have the defined meanings.
1. The term “thixotropic agent” means an agent which causes a suspension to exhibit a consistency that is viscous at rest, but fluid when agitated. Such a material has high static shear strength and low dynamic shear strength simultaneously.
2. The term “pharmaceutical agent” means an agent which has a therapeutic or palliative effect. Such agent is not restricted to those agents requiring a prescription for availability.

### DETAILED DESCRIPTION OF THE INVENTION

The aqueous suspensions of the invention comprises a pharmaceutical agent in particle form, a carrier for the pharmaceutical agent which includes a thixotropic agent and may also include bioacceptable additional ingredients and adjuvants, such as preservatives, emollients and the like.

Individual spray components may vary widely and particular pharmaceutical formulations are not the subject of this invention. Any particulate pharmaceutical agent which is capable of being suspended in an aqueous suspension may be used in the suspension. The pharmaceutical agents are present in amounts effective to provide the desired treatment to the body surfaces and/or tissues. Typical suspensions will contain pharmaceutical agents in amounts of from about 0.001 weight percent up to about 20 weight percent, more typically from about 0.001 weight percent to about 10 weight percent or less.

Numerous classes of useful pharmaceutical agents exist. Useful agents include but are not limited to, analgesic agents for pain relief such as opiods and non-steroidal anti-inflammatory agents, local anesthetics, antibiotics, hormones, steroids such as soluble cortisones, antihistamines, diruretics, vaccines and bone loss prevention agents. While some examples are listed herein, one skilled in the art will be aware of many more suitable pharmaceutical agents for use in the suspensions which are currently available or will become available when developed.

Examples of suitable analgesics include but are not limited to aceclofenac, acetaminophen, acetaminosalol, acetanilide, acetylsalicylsalicylic acid, alclofenac, alminoprofen, aloxiprin, aluminum bis(acetylsalicylate), aminoclorthenoxazin, aminopyrine, ammonium salicylate, antipyrine, antipyrine salicylate, antrafenine, apazone, aspirin, benoxaprofen, benzydamine, bernoprofen, calcium acetylsalicyate, fenoprofen, floctafenine, flufenamic acid, fluproquzone, flurbiprofen, imidazole salicylate, indoprofen, ketoprofen, ibuprofen, suprofen, talniflumate, tramadol, zomepirac, loxoprofen, and other analgesics known in the medical arts.

Examples of useful antibiotics include but are not limited to, third generation cephalosporins such as cefotaxine, moxolactam, cefoperazon, ceftizoxime, ceftazidime, ceftriaxone, cefitofur, and cefixime, penicillins such as amdinocillin, amoxicillin, bacampicillin, benzylpenicillinic acid, benzyl penicillin sodium, penicillin 0, penicillin V and derivatives thereof, macrolides such as eriythromycin, and derivates, dirithromycin, clarithromysine, and azithromycin, polypeptides such as amphomycin, bacitracin, and capreomycin, tetracyclines such as apicycline, clomocycline, clortetrocycline and the like, 2,4-diaminopyrimidines such as brodimoprim, quinolones and analogs thereof such as cinoxacin, ciprofloxacin, clinafloxacin, and flumequine, sulfones such as glucosulfone sodium, and other antibiotics known in the medical arts.

Examples of useful non-steroidal anti-inflammatory agents include, but are not limited to, aminoarylcarboxylic acid derivatives such as enfemamic acid, flufenamic acid, isosnixin, amfenac, ferofenamate, bufexamac, clopirac, fenclozic acid, and the like, arylbutryic acid derivatives, arylbutryic acid derivatives, arylpropionic acid derivatives, such as fenoprofen, ibuprofen, indoprofen, ketoprofen, naproxen oxaporzin, and the like, salicylic acid derivatives such as aspirin, phenyl salicylate, acetylsalicylate, and the like.

Examples of useful vaccines include but are not limited to those for HIV, flue, avian flu, polio, rubella and rubeola.

Examples of useful diuretics include furosemide, bumetanide, torsemide, ethacrynic acid, thiazides such as clorothiazide, hydrochlorothiazide, hydrofluormethazide, bendroflumethazide, nethyclothiazide, metolazone, polythiazide, Quinethazone and tichlormethiazide, potassium sparing diuretics such as spironolactone, triamterene, and amiloride, and others,

Examples of useful antihistamines include but are not limited to ethylenediamines such as pyrilamine and antazoline, ethanolamines such as diphenhydramine and doxylamine, alkylamines such as pheniramine, chlorpheniramine, dexclorpheniramine, and brompehmiramine, piperazines such as hydroxyzine, meclizine, and cyclizine, tricyclics such as cyproheptidine, azatadine, and promethazine, H1-receptor antagonists such as loratidine, fexofenadine, and other antihistamines known to the medical arts.

To prevent degradation of the pharmaceutical agent, one or more bioacceptable antioxidants can also be included. Useful antioxidants include sodium disulfate, ascorbic acid, sodium ascorbate, sodium thiosulfate and the like. When present, the antimicrobial agent typical comprises from about 0.001 weight percent to about 1 weight percent of the formulation.

For bio-stability of the suspension, an antimicrobial agent may be included in the formulation so long as it is bioacceptable. Examples of such agents include quaternary ammonium compounds such as bezalkonium chloride, mercurial agents such as thimerosal, alcohols such as benzyl alcohol, esters of parabenzoic acids, and other antimicrobial agents. When present, the antimicrobial agent typical comprises from about 0.001 weight percent to about 1 weight percent of the formulation.

Dispersing agents such as fatty alcohols and esters may also be present in the formulation such as polyoxyethylene oleates, commercially available under such names as Polysorbate™ 80.

Typical formulations for contact with body tissues and mucous membranes also may include some or all of the following: propylene glycol, polyethylene glycol, sodium phosphate monobasic, water, hydroxyethyl cellulose, sodium chloride and a buffering agent or sodium hydroxide or hydrochloric acid to adjust the pH.

The compositions may further include flavoring agents and sweetening agents including but not limited to, oil of peppermint, spearmint, wintergreen, clove, eucalyptus, cinnamon, lemon, lime and orange, cherry, sucrose, lactose, maltose, sorbitol, xylitol, sodium cyclamate, saccharine, and the like.

Formulations of the final thixotropic fluid media may also contain additional mixtures of such adjuvants as a food-grade vegetable oil, canola or safflower, dispersed in a mixture of a suitable fumed silica compounded to moisturize the nasopharynx subject to drying. In this situation, the fumed silica also acts as an emulsifying agent for the oil-water mixture. The addition of a medication may be combined together with a suitable hydrogel for slow release of the medication into the sprayed body surface. Importantly, as the resultant compound is sprayed through a calibration system as disclosed here or a standard manually operated spray bottle for nasal applications, droplets in the 50-200μ range should be produced such that they will remain on the mucosa of the nasopharynx and not pass into the bronchi.

Water is also present in the suspension, preferably purified water, such as distilled water, deoinized water, and the like. The amount of water present in the final suspension may be from 10 weight percent to about 98 weight percent of the formulation.

Approximate particle sizes useful in suspensions of the invention (references: Stanford Research Inst. Journal Vol. 5, 3^{rd} Quarter, 1961 and Ray, C D: Medical Engineering, Year Book Medical Publishers, 1974 p1213) include: Smoke dusts and fumes=0.01μ to 100μ (microns or micrometers) in diameter. Sprays, from 10μ upwards to 1000μ (1 mm). Nebulizer droplets, between 1μ and 20μ (about the size of a small human hair). The preferred droplet size should range around 1μ for medication to be carried by inhalation into the bronchi and lung bed, about 10μ to halt in the pharynx and bronchi and 10μ to 100μ that will lodge in nasal passages and nasopharynx. For an open tissue wound applications or for most lubricant or paint sprays, droplets ranging upwards to 500μ would be preferable. Highly viscous media or most organic gels are insufficiently fluid to be easily sprayed and then achieve the preferred higher viscosity on tissue contact. The more Newtonian or watery the medium, the easier it is to be sprayed. (Bulletin Cab-O-Sil® Properties and Functions, Cabot Corp. 9-2005, 8 pp.). While thixotropy can be found in certain gels it is more particularly characteristic of other synthetic substances, such as fumed silica which can be added to Newtonian fluids to achieve thixotropy.

Another desirable attribute for optimal delivery of the pharmaceutical agent and adjuvants by means of an atomizer or pump sprayer would be the use of a suspension medium composed in part by a hydrogel. Hydrogels and microencapsulated compounds are successfully used to controllably release contained or dissolved medication. The medication is dissolved and in part bonded within the complex of the hydrogel, and the medication may be in the form of a microencapsulation, then slowly dissolved or leached out at a rate determined by characteristics of the medication, the hydrogel or coating of the microcapsules. These characteristics are selectable, depending on the nature and formulation of the hydrogel and microcapsules, as part of the pharmaceutical manufacturing art. Appropriate ones of these agents may release hormones, various vascular active medication, vaccines and other drugs that are readily absorbed through the nasal membranes or other body surfaces. Fumed silica is FDA approved for use in foods and cosmetics. Some formulations for use on internal body parts may require additional study of bioacceptance.

As indicated above, the compounded material with the thixotropic agent increases the mutual molecular holding strength (viscosity) when the fluid to which it is added is essentially non-moving or slowly moving. Fumed silica exhibits this effect due to its peculiar molecular configuration having a great plurality of side projections that act in molecular dimensions as barbs or catching surfaces. As the fluid is more rapidly moved through the atomizer nozzle, the molecular barbs fail to hinder the motion and the viscosity declines. The characteristic thixotropy achieved is related to concentration of the fumed silica, the inherent viscosity of the fluid medium, the velocity of fluidic displacement, external forces such as gravity, surface tension and capillarity and pH of the medium, as well as the surface to which the medium is applied. Thixotropy increases with increasing acidity (falling pH). Most body surfaces, however, including mucous membranes, are at nearly neutral pH. A typical formulation for use in the nasopharyngeal passages might contain 0.25-1% fumed silica, by weight. When applied to the skin, the formulation may contain 1-2% fumed silica. For application to exposed body parts, the formulation may contain 0.5-1.5% fumed silica. Fumed silica is commercially available under such trademarks as Cab-o-Sil® from Cabot Corporation, and Aerosil® from DeGussa.

A number of water-soluble hydrogels may be used in certain preferred formulations of a novel medium. Gelatins, agars and other cellulosics, e.g., organic polysaccharides; inorganic polymers (polyvinyl chloride, polyacrylonitrile, ethylene oxide, and certain water-soluble waxes) may be used. A typical formulation for use on or in body surfaces may consist of a water-soluble hydrogel, e.g., polyacrylonitrile in an aqueous suspension of 1-5%, by weight. In that the mutual presence of various components as above indicated as well as both fumed silica and a hydrogel will exert combined influences on the real and apparent viscosity, the combinations must be optimized, which can be performed only by trial and error or a spray calibration device as disclosed in a parallel application.

In that an optimization process for mixture control is required, precise singular or plural thixotropic formulations need not be recited in this application. One skilled in the art will be able to perform controlled calibration tests on individual formulations in order to optimize a specific compounded thixotropic medium. In addition, trial and error experimentation can be employed for medium optimization. In keeping with the above, typical formulations are moderately but not precisely detailed herein.

### EXAMPLE OF USE

When applied to the nasopharyngeal tracts, the preferred, optimized formulation containing the prescribed medications or non-prescription materials (such as a simple moistening agent or an anti-drying oil) is loaded into an optimized atomizer and the bulb of the atomizer is firmly squeezed in the upright direction into the nares, dispensing the medium. The user may be instructed to inhale (or not, with or without the mouth open) during this maneuver. For application on surgically exposed body surfaces (such as where an anti-inflammatory drug, e.g., cortisone or local anesthetic may be applied prior to tissue closure, or another agent, such as a protecting film, oil or polymer, may be applied), a sterile atomizer and contained medium would be used. The atomizer may require further optimization for use in the horizontal plane or when inverted over the exposed tissues.

Although specific and general embodiments have been described herein, it will be appreciated by those of ordinary skill in the art that a variety of alternate and/or equivalent implementations may be substituted for the specific embodiments described without departing from the scope of the present invention. This application is intended to cover any adaptations or variations of the specific embodiments discussed herein. Therefore, it is intended that this invention be limited only by the claims and the equivalents thereof.

What is claimed is:

1. An aqueous fluid suspension for delivery of a pharmaceutical agent, said suspension having a sheared viscosity and an unsheared viscosity, wherein said sheared viscosity is less than about 200 centipoise, the suspension being sprayable when sheared, and wherein said unsheared viscosity is greater than about 400 centipoise, said suspension comprising said pharmaceutical agent and a carrier therefor, said carrier comprising a thixotropic agent.

2. The composition of claim 1 wherein said aqueous suspension is delivered in droplets having an average droplet size of from about 10 microns to about 100 microns.

3. The composition of claim 1, wherein the thixotropic agent includes fumed silica.

4. The composition of claim 1 wherein said carrier is a hydrogel.

5. The composition of claim 4 wherein said hydrogel encapsulates said pharmaceutical agent.

6. The suspension of claim 3 wherein said hydrogel comprises water and at least one hydrophilic polymer.

7. The composition of claim 1 wherein said pharmaceutical agent is selected from the group consisting of pain relief agents, antibiotics, hormones, soluble cortisones, antihistamines, diruretics, vaccines and bone loss prevention agents.

8. The suspension of claim 5 wherein the pharmaceutical agent is selected from the group consisting of third generation cephalosporins, soluble cortisones, and antihistamines.

9. The suspension of claim 1 wherein said suspension further comprises an emollient and surface evaporation retardant comprising an inert bioacceptable oil selected from the group consisting of vegetable oil, canola oil, sesame oil and corn oil.

10. An aqueous fluid suspension for delivery of a pharmaceutical agent, said suspension having a sheared viscosity and an unsheared viscosity, wherein said sheared viscosity is less than about 200 centipoise, the suspension being sprayable when sheared, and wherein said unsheared viscosity is at least about 5 times greater than said sheared viscosity, and the suspension returns to its unsheared viscosity within about 20 seconds after shearing, the suspension comprising said pharmaceutical agent and a carrier therefore, said carrier comprising a thixotropic agent.

11. The composition of claim 10 wherein said aqueous suspension is delivered in droplets having an average droplet size of from about 10 microns to about 100 microns.

12. The composition of claim 10, wherein the thixotropic agent includes fumed silica.

13. The composition of claim 1 wherein said carrier is a hydrogel.

14. The composition of claim 13 wherein said hydrogel encapsulates said pharmaceutical agent.

15. The suspension of claim 13 wherein said hydrogel comprises water and at least one hydrophilic polymer.

16. The composition of claim 10 wherein said pharmaceutical agent is selected from the group consisting of antibiotics, hormones, soluble cortisones, antihistamines, diruretics, vaccines and bone loss prevention agents.

17. The suspension of claim 16 wherein the pharmaceutical agent is selected from the group consisting of third generation cephalosporins, soluble cortisones, and antihistamines.

18. The suspension of claim 10 wherein said suspension further comprises an emollient and surface evaporation retardant comprising an inert bioacceptable oil.

19. The suspension of claim 18 wherein said inert bioacceptable oil is selected from the group consisting of vegetable oil, canola oil, sesame oil and corn oil.
